(19) United States
(12) Patent Application Publication
(10) Pub. No.: US 2007/0298117 A1
(43) Pub. Date: Dec. 27, 2007

(54) Compositions and kits comprising a melatonin component and a chondroprotective component

(75) Inventors: Jesus Velazquez, West Chester, OH (US); Amy Violet Trejo, Oregonia, OH (US); James Patrick Ebel, Lebanon, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/820,388

(22) Filed: Jun. 19, 2007

Related U.S. Application Data
(60) Provisional application No. 60/816,074, filed on Jun. 23, 2006.

(51) Int. Cl. A61K 31/403; A61K 31/726; A61K 35/32; A61K 38/16; A61P 43/00

(52) U.S. Cl. 424/548; 514/12; 514/415; 514/54

(57) ABSTRACT

Disclosed herein are compositions and kits, wherein each composition comprises a melatonin component, a chondroprotective component, or both, and wherein each kit comprises a melatonin component and a chondroprotective component. The compositions and kits are useful for restorative sleep function and restorative joint structure or restorative joint function. Further disclosed herein are methods of using the compositions and kits.

### CROSS REFERENCE TO RELATED APPLICATIONS

This application claims the benefit of U.S. Provisional Application No. 60/816,074, filed Jun. 23, 2006.

### FIELD OF THE INVENTION

The present invention is directed to compositions and kits, wherein each composition comprises a melatonin component, a chondroprotective component, or both, and wherein each kit comprises a melatonin component and a chondroprotective component. The compositions and kits are useful for restorative sleep function and restorative joint structure or restorative joint function. The invention is further directed to methods of using the compositions and kits.

### BACKGROUND OF THE INVENTION

One of the functions of sleep is the maintenance, restoration, and repair of the body. Sleep is generally characterized by anabolic activity (including building and remodeling) in muscle, bone, connective tissue, skin, and major organs including the brain. One result of this activity is restoration of function including physical and mental performance such as stamina, energy, and mental alertness.

Supplements, foods, medicines, and other products that are used to induce and improve sleep with respect to duration and quality are commercially available. Examples are supplements such as melatonin, valerian and hops, over-the-counter medications like diphenhydramine and doxylamine, and prescription medications such as AMBIEN® and LUNESTA®.

However, these products fail to provide any additional benefits beyond induction and improvement of sleep, and the indirect benefits derived from such induction and improvement. It would be beneficial to utilize the sleep cycle to more directly induce these benefits, when the body is susceptible to building and remodeling activities, such that the benefits are achieved and an even greater level than would otherwise be achieved.

### SUMMARY OF THE INVENTION

The present invention is directed to compositions and kits useful for restorative sleep function and restorative joint structure or restorative joint function.

In particular, the invention is directed to a composition comprising:
(a) a melatonin component; and
(b) a chondroprotective component.

The invention is further directed to a kit comprising:
(a) a composition comprising a melatonin component; and
(b) a composition comprising a chondroprotective component.

The invention is further directed to methods of using the compositions and kits.

### DETAILED DESCRIPTION OF THE INVENTION

The present invention is directed to combinations of a melatonin component and a chondroprotective component, whether such components are combined in a single composition or in discrete compositions within a kit. The compositions and kits, and methods of use thereof, are useful for more directly inducing the benefits of restorative sleep, particularly in the improvement of joint structure and function.

Various documents including, for example, publications and patents, are recited throughout this disclosure. All such documents are hereby incorporated by reference.

Trade names for products or components including various ingredients may be referenced herein. The inventors herein do not intend to be limited by materials under a certain trade name.

In the description of the invention various embodiments or individual features are disclosed. As will be apparent to the ordinarily skilled practitioner, all combinations of such embodiments and features are possible and can result in preferred executions of the present invention.

The compositions herein may comprise, consist essentially of, or consist of any of the elements as described herein.

While various embodiments and individual features of the present invention have been illustrated and described, various other changes and modifications can be made without departing from the spirit and scope of the invention. As will also be apparent, all combinations of the embodiments and features taught in the foregoing disclosure are possible and can result in preferred executions of the invention.

With respect to dosing preferences, dosage levels are developed based on typical human subjects (e.g., a 65 kg subject). Wherein the present composition is used in other mammals or in various human subjects, it may be necessary to modify the dosage. Modification of dosages based on the needs of the subject is well within the skill of the ordinary artisan. It is therefore understood that these dosage ranges are by way of example only, and that daily administration can be adjusted depending on various factors. The specific dosage of the compound to be administered, and the duration of treatment are interdependent. The dosage and treatment regimen will also depend upon such factors as the specific compound used, the treatment indication, the efficacy of the compound, the personal attributes of the subject (such as, for example, weight, age, gender, and medical condition of the subject), and compliance with the treatment regimen.

The present invention is directed to compositions and kits, wherein each composition comprises a melatonin component, a chondroprotective component, or both, and wherein each kit comprises a melatonin component and a chondroprotective component. The compositions and kits are useful for restorative sleep function and restorative joint structure or function.

The melatonin component and the chondroprotective component are described as follows. These compounds may be utilized together in a composition, or may be provided in separate compositions as part of a kit. In particular, dosing convenience may be provided in embodiments wherein each of these compounds is present in one composition, while convenience and further dosing flexibility may be provided wherein each of these compounds is present in separate compositions as part of the kit. For example, the user of the kit may be in need of only one of the melatonin component and the chondroprotective component on any given day, and therefore excess dosing of unnecessary compounds during administration may be avoided.

The melatonin component and the chondroprotective component are described as follows. In addition, optional dosage guidance is provided, as well as optional adjuncts and dose forms.

### The Melatonin Component

The present kits and compositions comprise a melatonin component. As used herein, the melatonin component includes melatonin, melatonin precursors, melatonin agonists, and compounds that raise endogenous melatonin levels, as well as mixtures thereof. See e.g., U.S. Patent Publication 2004/0044064.

Melatonin components, including melatonin, are commercially available. Non-limiting examples of melatonin precursors, melatonin agonists; and such other compounds that mimic melatonin activity, include N-acetyl-5-hydroxytryptamine. For example, these may include compounds that compete with melatonin at the melatonin receptor and compounds that stimulate melatonin receptors to have an effective opposite to that of melatonin (melatonoin inverse agonists), in addition to drugs (melatonin blockers or melatonin stimulants) and interventions (such as exposure to light or darkness) that lower or raise, respectively, endogenous melatonin levels.

In one embodiment herein, the melatonin component is melatonin.

In one embodiment herein, a composition comprising a melatonin component comprises a dosage of about 0.01 mg to about 100 mg the melatonin component, alternatively from about 0.1 mg to about, 10 mg of the melatonin component, and alternatively from about 0.1 mg to about 1 mg of the melatonin component. The ordinarily skilled artisan will adjust the dose to effect the desired change in phase of the circadian rhythm of endogenous melatonin production.

Since melatonin components, particularly melatonin, may be absorbed across almost all tissues, many routes of administration are possible. These include but are not limited to submucosal, sublingual, intranasal, ocular cul-de-sac, rectal, transdermal, buccal, intravenous, intramuscular, and subcutaneous routes of administration. A variety of administration means, including but not limited to capsules, tablets, suppositories, or any reservoir capable of containing and dispensing melatonin, are useful. In a preferred embodiment herein, the composition comprising the melatonin component is administered orally.

Typically, the composition comprising the melatonin component is administered in a manner commensurate with desired onset of sleep. In one embodiment, the composition comprising the melatonin component is administered within 1 hour of desired sleep time, at least about 1 hour of desired sleep time, at least about 4 hours prior to desired sleep time, or at least about 8 hours prior to desired sleep time. Compositions in sustained or delayed release form may typically be administered at least about 4 hours or at least about 8 hours prior to desired sleep time. In one embodiment, administration preferably follows the descriptions set forth in the following documents: U.S. Pat. Nos. 5,242,941; 5,420,152; 5,591,768; 5,707,652; 5,716,978; 6,069,164; 6,423,738; 6,638,963; and 6,794,407; and U.S. Patent Publication Nos. 2004/0044064 and 2003/0008912.

### The Chondroprotective Component

The chondroprotective component utilized herein may be any compound that provides joint health, bone health, and/or anti-inflammation, as described above. Chondroprotective components are quite well-known in the art, and the ordinarily skilled artisan has the capability to choose any such compound for use in the present invention.

Without intending to be limited by theory, the chondroprotective component is important for enhancing joint function as the component aids in the stimulation of proteoglycan and collagen in vivo. Proteoglycan provides the connective tissue, for example, collagen, which is necessary for joint health. Indeed, proteoglycan is comprised of glycosaminoglycans (often termed “GAGs”) which are long chains of modified sugars. Aminosugars and methylsulfonylmethane are useful for building glycosaminoglycans and proteoglycan.

Non-limiting examples of chondroprotective components which are useful herein include gelatin, cartilage, aminosugars, glycosaminoglycans, methylsulfonylmethane, precursors of methylsulfonylmethane, S-adenosylmethionine, salts thereof, and mixtures thereof. Preferably, the chondroprotective component is selected from gelatin, cartilage, aminosugars, glycosaminoglycans, S-adenosylmethionine, salts thereof, and mixtures thereof. More preferably, the chondroprotective component is selected from aminosugars, glycosaminoglycans, S-adenosylmethionine, salts thereof, and mixtures thereof. Even more preferably, the chondroprotective component is selected from aminosugars, glycosaminoglycans, salts thereof, and mixtures thereof. Most preferably, the chondroprotective component is a salt of an aminosugar, particularly wherein the aminosugar is glucosamine.

Examples of these chondroprotective components, and preferred embodiments thereof, are described in expanded detail as follows.

### Gelatin

As is commonly known, gelatin is a protein obtained from the partial hydrolysis of collagen, which is the major structural and connective protein tissue in mammals. Gelatin typically contains from about 84% to about 90% protein, from about 1% to about 2% mineral salts, and from about 8% to about 15% water (these are non-limiting approximations). Gelatin typically contains specific amounts of 18 different amino acids, which are joined together to form polypeptide chains of approximately 1,000 amino acid residues per chain.

Typically, the collagen obtained for gelatin production is from animal bones and skins, e.g., from cows and pigs. Gelatin production will typically involve the subjection of collagenous material to alkaline pre-treatment, followed by hot-water extraction (providing gelatin having an iso-electric point of about 5). Acidic pre-treatment may also be utilized (providing gelatin having an iso-electric point of from about 7 to 9).

In accordance with the present invention, wherein gelatin is included within a present composition, a single dose of gelatin within the composition is preferably from about 1 mg to about 2000 mg, more preferably from about 100 mg to about 700 mg, even more preferably from about 150 mg to about 600 mg, and most preferably from about 200 mg to about 400 mg. Typically, the composition comprising the gelatin could be dosed from about once to about five times daily, preferably from about once to about three times daily, most preferably once daily.

### Cartilage

Cartilage may be chosen as the chondroprotective component in the present compositions. As is commonly known in the art, cartilage is a tough, elastic tissue present in the joints (as well as other locations) of the bodies of various mammals. Cartilage is comprised of at least one of calcium, proteins, carbohydate mucopolysaccharides (e.g., chondroitin), and collagen.

Particularly preferred for use herein is bovine cartilage and shark cartilage. Bovine cartilage is primarily derived from the trachea of cows (also known as bovine tracheal cartilage, or BTC). It is similar in structure to shark cartilage. Shark cartilage is a widely utilized cartilage source, as the skeletons of sharks are primarily composed of cartilage rather than bone.

In accordance with the present invention, wherein cartilage is included within a present composition, a single dose of cartilage within the composition is preferably from about 1 mg to about 2000 mg, more preferably from about 100 mg to about 700 mg, even more preferably from about 150 mg to about 600 mg, and most preferably from about 200 mg to about 400 mg. Typically, the composition comprising the cartilage is dosed from about once to about five times daily, preferably from about once to about three times daily, most preferably once daily.

### Aminosugars

One or more aminosugars may be chosen as the chondroprotective component herein. The aminosugars are monosaccharide components (i.e., hexoses) which are modified with an amine functionality. The amine functionality may be a free amine moiety or a protected amine moiety (e.g., N-acetyl amine). Preferably, the aminosugar is a precursor to glycosaminoglycan, which is important for construction of joint constituents (e.g., collagen). Additionally, certain aminosugars may serve to inhibit the activity of enzymes which are implicated in breakdown the cartilage in osteoarthritics (e.g., mannosamine, which has been discovered to inhibit aggrecanase). The aminosugars are well-known in the art; many aminosugars are naturally occurring. Particularly preferred aminosugars include glucosamine, salts of glucosamine, galactosamine, salts of galactosamine, mannosamine, salts of mannosamine, as well as the N-acetyl derivatives of the foregoing, including N-acetyl glucosamine and N-acetyl galactosamine. More preferably, the aminosugars include glucosamine and salts of glucosamine, most preferably salts of glucosamine.

Particularly preferred salts of glucosamine include glucosamine sulfate and glucosamine hydrochloride. The salts of glucosamine are particularly preferred to aid bioavailability to the aminosugar in addition to the bioavailability benefit achieved by the second component (as described herein below).

As an example, glucosamine provides the building block needed in vivo to manufacture glycosaminoglycan, which is found in cartilage. Thus, glucosamine, and other aminosugars, function not only to relieve symptoms of joint pain but also inhibits, stops, and/or reverses the degenerative process.

Typical single dosing of the aminosugars is preferably from about 1 mg to about 5000 mg, more preferably from about 100 mg to about 3600 mg, even more preferably from about 150 mg to about 2200 mg, and most preferably from about 250 mg to about 1900 mg, based on the molecular weight of glucosamine hydrochloride. For example, a particularly preferred dosage of glucosamine hydrochloride is about 1800 mg, which translates to about 1480 mg of glucosamine. All other aminosugars may be similarly dosed, based on the molecular weight of glucosamine hydrochloride. Typically, the composition comprising the aminosugar is dosed from about once to about five times daily, preferably from about once to about three times daily, most preferably once daily.

### Glycosaminoglycans

One or more glycosaminoglycans may be utilized as the chondroprotective component herein. The glycosaminoglycans are commonly known as GAGs, and are precursors to joint structure, for example, collagen. The glycosaminoglycans may also be important for the healing of bone.

Suitable glycosaminoglycans will be well-known to the ordinarily skilled artisan. Preferred glycosaminoglycans include chondroitin, hyaluronic acid, keratan, heparin, and dermatin, as well as salts of the foregoing. For example, chondroitin sulfate is a particularly preferred chondroitin salt. As with the aminosugars, salts of the glycosaminoglycans are particularly preferred for use herein.

As an example, chondroitin provides the structure and allows various molecules to transport through cartilage (which is important, since there is no blood supply to cartilage). Chondroitin is a major constituent of cartilage and contains repeating chains of mucopolysaccharides.

Typical single dosing of the glycosaminoglycans is preferably from about 1 mg to about 10 grams, more preferably from about 100 mg to about 5 grams, even more preferably from about 150 mg to about 1000 mg, and most preferably from about 250 mg to about 800 mg, based on the molecular weight of chondroitin. All other glycosaminoglycans may be similarly dosed, based on the molecular weight of chondroitin. Typically, the composition comprising the glycosaminoglycan is dosed from about once to about five times daily, preferably from about once to about three times daily, most preferably once daily.

### Methylsulfonylmethane and Precursors of Methylsulfonylmethane

The chondroprotective component herein may also be methylsulfonylmethane, or a precursor thereof. As used herein, the term “precursor thereof” means a compound which, in mammalian systems, is converted to methylsulfonylmethane in vivo. Methylsulfonylmethane, and precursors thereof, are common ingredients found in vivo and in nature, e.g., in unprocessed foods. Without intending to be limited by theory, it is believed that the sulfur moiety present in methylsulfonylmethane, and its precursors, provides the disulfide bridging (also commonly known as “tie-bars” or “cross-links”) necessary to hold the connective tissue in joints together.

While unprocessed foods contain-methylsulfonylmethane, and the precursors thereof, conventional food processing and preparation causes the loss of these compounds from the foods. Therefore, commonly ingested foods may become deficient in these compounds. In these respects, methylsulfonylmethane is similar to vitamins and minerals which are typically partially or totally lost during normal food processing and preparation. It is therefore an important embodiment of this invention to include methylsulfonylmethane or a precursor thereof in the present compositions.

Non-limiting examples of precursors of methylsulfonylmethane include methionine and methyl sulfide. See e.g., Herschler et al., U.S. Pat. No. 4,863,748, issued Sep. 5, 1989. Precursors of methylsulfonylmethane are associated with a variety of health benefits, including joint benefits (such as relief from osteoarthritis and rheumatoid arthritis), as well as anti-inflammation.

In accordance with the present invention, wherein methanesulfonylmethane is included within a present composition, a single dose of methanesulfonylmethane within the composition is preferably from about 0.01 mg to about 2000 mg, more preferably from about 0.01 mg to about 500 mg, even more preferably from about 1 mg to about 200 mg, and most preferably from about 1 mg to about 100 mg. The precursors of methanesulfonylmethane may be similarly dosed, based on the molecular weights of the precursors relative to methanesulfonylmethane. Typically, the composition comprising methylsulfonyl methane, or a precursor thereof, is dosed from about once to about five times daily, preferably from about once to about three times daily, most preferably once daily.

### S-Adenosylmethionine

S-adenosylmethionine, which is commonly known as SAM-e, is a compound which is found in most, if not all, living cells. Without intending to be limited by theory, SAM-e is produced through reaction of the essential amino acid methionine and the energy molecule known as adenosine triphosphate (commonly known as ATP). SAM-e manufactures the components of cartilage and repairs, restores, and maintains joint function. SAM-e is made in vivo from the amino acid methionine, and is found in ordinary dietary sources such as meats, soybeans, eggs, seeds, and lentils.

In accordance with the present invention, wherein SAM-e is included within a present composition, a single dose of SAM-e within the composition is preferably from about 1 mg to about 2000 mg, more preferably from about 100 mg to about 700 mg, even more preferably from about 150 mg to about 600 mg, and most preferably from about 200 mg to about 400 mg. Typically, the composition comprising SAM-e is dosed from about once to about five times daily, preferably from about once to about three times daily, most preferably once daily.

### Kits of the Present Invention

In one embodiment herein, the kits comprise a composition comprising the melatonin component and a composition comprising the chondroprotective component. In this embodiment, the kits comprise at least two discrete compositions, i.e., at least two compositions that are compositionally distinct.

In this embodiment, the kits are particularly advantageous to different needs regarding time of dosing of the compositions. For example, it may be advantageous to administer the composition comprising the melatonin composition at a time relative to desired sleep time of the mammal, for example, within about 1 hour, at least about 1 hour, at least about 4 hours or at least about 8 hours prior to desired sleep time. Such time of administration may be dependent upon a variety of factors, such as whether the composition is formulated as an immediate, sustained, or delayed release formulation, or dependent upon the particular restorative sleep needs of the mammal. On the other hand, time of administration of the composition comprising the chondroprotective component may not be as important, and therefore this composition may be, for example, administered at any time that is convenient to the mammal. Of course, the invention fully contemplates concurrent administration of both of these compositions, which may be particularly convenient for the mammal, but the kit allows ease of flexibility and therefore greater opportunity for enhanced compliance with a treatment regimen.

The kits herein may be packaged in any manner, such as any manner that is ultimately convenient for the mammal. For example, the kit may offer a plurality of blister packs wherein each blister pack contains a daily dose of the composition comprising the melatonin component and the composition comprising the chondroprotective component. In this example, the compositions may be formulated as capsules or tablets, and each blister pack may contain one or more of each type of composition, depending upon the frequency of daily dose. Weekly, monthly, or other types of kits offering multiple doses of the discrete compositions may be provided.

### Methods of Use

The methods of the present invention comprise orally administering (i.e., through ingestion) a composition of the present invention to a mammal, preferably a human, to provide various health benefits, including inducing restorative sleep function, inducing restorative joint function, inducing restorative joint structure, and combinations thereof. The compositions of the present invention are most preferably ingested by consumers primarily desiring restorative sleep function and further desiring to complement this benefit with induction of restorative joint function or structure while taking advantage of the restorative actions of the mammalian body during rest sleep. The compositions are also preferably ingested by consumers who experience joint and/or bone dysfunction or those who desire to maintain current joint and/or bone function (i.e., prophylacetic use). The compositions of this invention may also be ingested as a supplement to normal dietetic requirements. Frequency of administration is not limited, however, such administration is typically at least once weekly, more preferably at least 3 times weekly, and most preferably at least once daily. Extent of need of restorative sleep function may dictate administration of at least a composition comprising the melatonin component.

As used herein, “restorative sleep function” refers to alleviation of any circadian rhythm phase-shifting effect, jet lag, winter depression, shift work-related desynchronies, sleep phase disorders, and other sleep disorders, improvement in sleep quality, improvement of sleep duration, and combinations thereof.

As used herein, “restorative joint function” refers to alleviation of any conditions associated with the joint, such as for example soreness, stiffness, and the like. As used herein, “restorative joint structure” refers to repair or maintenance of the joint or surrounding structure such as cartilage or collagenous tissues.

As used herein, the term “orally administering” with respect to the mammal (preferably, human) means that the mammal ingests or is directed to ingest (preferably, for the purpose of providing one or more of the health benefits described herein) one or more compositions of the present invention. In one embodiment, the composition is formulated as a tablet, capsule, food or beverage composition. Wherein the mammal is directed to ingest one or more of the compositions, such direction may be that which instructs and/or informs the user that use of the composition may and/or will provide one or more general health and/or general physiological benefits including, but not limited to, restorative sleep function, inducing restorative joint function, inducing restorative joint structure, and combinations thereof. For example, such direction may be oral direction (e.g., through oral instruction from, for example, a physician, health professional, sales professional or organization, and/or radio or television media (i.e., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or packaging associated with the composition (e.g., a label present on a package containing the composition). As used herein, “written” means through words, pictures, symbols, and/or other visible descriptors. Such direction need not utilize the actual words used herein, for example, “sleep”, “restorative” “joint”, “bone”, “human”, or “mammal”, but rather use of words, pictures, symbols, and the like conveying the same or similar meaning are contemplated within the scope of this invention.

Since melatonin appears to be absorbed across almost all tissues, many routes of administration are possible. These include but are not limited to submucosal, sublingual, intranasal, ocular cul-de-sac, rectal, transdermal, buccal, intravenous, intramuscular, and subcutaneous routes of administration. A variety of administration means, including but not limited to capsules, tablets, suppositories, or any reservoir capable of containing and dispensing melatonin, are useful. In a preferred embodiment herein, the melatonin is administered orally.

The compositions herein may be, for example, formulated as an immediate or sustained release formulation. In one embodiment, a composition containing melatonin is in immediate release form. In another embodiment, a composition containing melatonin is in sustained release form (such as wherein the melatonin is continuously released over a set period of time). In yet another embodiment, a composition containing melatonin is in delayed release form (such as wherein the melatonin released at some time after administration). Wherein the invention is presented as a kit, the composition containing the chondroprotective component may be, for example, formulated as an immediate release composition, even wherein the composition containing the melatonin is formulated as a sustained release or delayed release formulation.

Further, the methods of the invention relate to the timing of the administration of the dosage of melatonin to the mammal. The timing of the melatonin in the mammal as described results in a specific phase shift (phase advance or phase delay) in the mammal's circadian rhythms.

### EXAMPLES

The following are non-limiting examples of the present compositions which are prepared utilizing conventional methods. The following examples are provided to illustrate the invention and are not intended to limit the scope thereof in any manner.

### Example 1

A kit is prepared, comprising a blister pack intended for seven day use. The blister pack contains seven compositions comprising a melatonin component and seven compositions comprising a chondroprotective component. All compositions are in tablet form.

The compositions comprising the melatonin component are in sustained release form and contain the following ingredients, at the indicated amounts:

| Component | (Wt %, approximate) |
|---|---|
| Melatonin | 0.5 |
| Lactose | 59 |
| Hydroxymethylcellulose | 30 |
| Microcrystalline Cellulose | 10 |
| Magnesium Stearate | 0.5 |

The compositions comprising the chondroprotective component contain:

| Component | Weight |
|---|---|
| Glucosamine Hydrochloride | 1500 mg |

Each day over a seven day period, a human in need of restorative sleep function and having soreness of joints ingests one tablet of the composition comprising the melatonin component and one tablet of the composition comprising the chondroprotective component. The human ingests the composition comprising the melatonin component about 4 hours prior to 11:00 PM, which is this particular human's desired sleep time. The human ingests the composition comprising the chondroprotective component during any convenient time of day, which is flexible throughout the dosing regimen. The human obtains additional kits and administers the compositions daily over an indefinite period of time.

### Example 2

A kit is prepared as in Example 1, except the composition comprising the melatonin component is a delayed release formulation. The composition comprising the melatonin component is in the form of a tablet, which is enterically coated with a methacrylic acid copolymer and simethicone mixture. The coating has the following approximate formula:

| Component | (Wt %, approximate) |
|---|---|
| Methacrylic Acid Copolymer | 99.85 |
| Simethicone | 0.15 |

Each day over a seven day period, a human in need of restorative sleep function and having soreness of joints ingests one tablet of the composition comprising the melatonin component and one tablet of the composition comprising the chondroprotective component. The human ingests the composition comprising the melatonin component about 8 hours prior to 11:00 PM, which is this particular human's desired sleep time. The human ingests the composition comprising the chondroprotective component during any convenient time of day, which is flexible throughout the dosing regimen. The human obtains additional kits and administers the compositions daily over an indefinite period of time.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising:
(a) a melatonin component; and
(b) a chondroprotective component.

2. The composition according to claim 1 wherein the melatonin component is melatonin.

3. The composition according to claim 2 wherein the chondroprotective component is selected from the group consisting of gelatin, cartilage, aminosugars, glycosaminoglycans, methylsulfonylmethane, precursors of methylsulfonylmethane, S-adenosylmethionine, salts thereof, and mixtures thereof.

4. The composition according to claim 3 wherein the chondroprotective component is selected from the group consisting of glucosamine, salts thereof, and mixtures thereof.

5. The composition according to claim 4 comprising from about 0.01 mg to about 100 mg of melatonin.

6. The composition according to claim 5 comprising from about 1 mg to about 5000 mg of the chondroprotective component.

7. The composition according to claim 5 which is selected from the group consisting of tablets and capsules.

8. A kit comprising:
(a) a composition comprising a melatonin component; and
(b) a composition comprising a chondroprotective component.

9. The kit according to claim 8 wherein the melatonin component is melatonin.

10. The kit according to claim 8 wherein the chondroprotective component is selected from the group consisting of gelatin, cartilage, aminosugars, glycosaminoglycans, methylsulfonylmethane, precursors of methylsulfonylmethane, S-adenosylmethionine, salts thereof, and mixtures thereof.

11. The kit according to claim 8 wherein the chondroprotective component is selected from the group consisting of glucosamine, salts thereof, and mixtures thereof.

12. The kit according to claim 8 wherein the composition comprising the melatonin component comprises from about 0.01 mg to about 100 mg of melatonin.

13. The kit according to claim 8 wherein the composition comprising the chondroprotective component comprises from about 1 mg to about 5000 mg of the chondroprotective component.

14. The kit according to claim 13 which each composition is, independently, selected from the group consisting of tablets and capsules.

15. A method selected from inducing restorative sleep function, inducing restorative joint function, inducing restorative joint structure, and combinations thereof comprising administering to a mammal in need thereof the composition according to claim 1.

16. The method according to claim 15 wherein the administration is oral.

17. The method according to claim 16 wherein the composition is administered at least about 4 hours prior to desired sleep time of the mammal.

18. The method according to claim 17 wherein the composition is in sustained release form.

19. The method according to claim 18 wherein the composition is administered at least about 8 hours prior to desired sleep time of the mammal.

20. A method selected from inducing restorative sleep function, inducing restorative joint function, inducing restorative joint structure, and combinations thereof comprising administering to a mammal in need thereof the composition comprising the melatonin component and the composition comprising the chondroprotective component.

21. The method according to claim 20 wherein the administration is oral.

22. The method according to claim 21 wherein the composition comprising the melatonin is administered at least about 4 hours prior to desired sleep time of the mammal.

23. The method according to claim 22 wherein the composition comprising the melatonin component is in sustained release form.

24. The method according to claim 23 wherein the composition comprising the melatonin component is administered at least about 8 hours prior to desired sleep time of the mammal.
